(19) United States
(12) Patent Application Publication
(10) Pub. No.: US 2005/0250791 A1
(43) Pub. Date: Nov. 10, 2005

(54) Process for preparing monoarylpiperazines

(75) Inventor: Ulrich Scholz, Mulheim (DE)

Correspondence Address: LANXESS CORPORATION, 111 RIDC PARK WEST DRIVE, PITTSBURGH, PA, 15275-1112, US

(73) Assignee: LANXESS Deutschland GmbH

(21) Appl. No.: 11/120,488

(22) Filed: May 3, 2005

(30) Foreign Application Priority Data
May 5, 2004 (DE) 1020040227659

(51) Int. Cl. A61K 31/495; C07D 241/04

(52) U.S. Cl. 514/255.03; 544/392

(57) ABSTRACT

The invention provides a process for preparing arylpiperazines from the corresponding aryl chlorides or bromides and piperazine using a base and a catalyst consisting of a palladium salt and a bisaryldialkylphosphine.

The invention provides a process for preparing arylpiperazines from the corresponding aryl chlorides or bromides and piperazine using a base and a catalyst consisting of a palladium salt and a bisaryldialkylphosphine.

Monoarylpiperazines find use in particular as building blocks for preparing active pharmaceutical ingredients.

It is known that monoarylpiperazines can be prepared starting from anilines by reaction with activated diethylenamines, for example di(chloroethyl)amine. A disadvantage of this process is the fact that only few highly substituted anilines are commercially available and this process is thus economically viable only for few monoarylpiperazines.

It is also known from E. Brenner, R. Schneider, Y. Fort, Tetrahedron 58, (2002), 6913-6924 that monoarylpiperazines can be prepared by reacting chloroaromatics and piperazines using a catalyst, the catalyst consisting of nickel acetate and 2,2′-bipyridine. A disadvantage of this process is the fact that the selectivity of mono-relative to diarylation of 85:15 in these reactions is too low for industrial purposes, and additionally that disproportionately large amounts of 10 mol % of the toxic nickel acetate have to be used.

The reaction of substituted aryl halides with piperazine using a palladium-triarylphosphine catalyst was described for the first time by Zhao (S. Zhao, A. K. Miller, J. Berger, L. A. Flippin, Tetrahedron Letters, 37 (1997), 4463-4466). However, this process likewise requires large amounts of catalyst of 2 to 5 mol % in order to achieve acceptable chemical yields. Also disadvantageous is the fact that high selectivities in the formation of monoarylpiperazines in comparison to diarylpiperazines can be achieved only when a very high excess of 4 to 6 equivalents of piperazine is added, which both complicates the workup of the reaction and leads to higher costs for the performance of the reaction.

The selectivity of the reaction may be increased by using monoprotected piperazine in the reaction (F. Kerrigan, C. Martin, G. H. Thomas, Tetrahedron Letters, 39 (1998), 2219-2222). However, as a result of the preparation of the protected piperazine and the subsequent removal of the protecting group, this measure requires two undesired additional process steps which make the overall process uneconomic.

Acceptable yields using small amounts of catalyst have been achieved by using a palladium-trisalkylphosphine catalyst (M. Nishiyama, Y. Koie, EP 0 802 173). However, a disadvantage of this process is that a sixfold excess of piperazine has to be used. In addition, this process is carried out using a highly air-sensitive and self-igniting phosphine, so that the economic viability of the process suffers as a result of additionally required safety measures. In addition, this process often only succeeds when the expensive aryl bromides are used.

The reaction of aryl chlorides and piperazine using a catalyst consisting of air-stable N-heterocyclic carbenes and a palladium salt, and also a strong alkoxide base, has likewise already been described (S. R. Stauffer, S. Lee, J. P. Stambuli, S. I. Hauk, J. F. Hartwig, Organic Letters 2 (10), 2000, 1423-1426), but it is expressly mentioned here for the preparation of arylpiperazines that large amounts of catalyst of 2 mol % are necessary. In this process too, a very high excess of piperazine is necessary.

There is accordingly a need to provide a process by which a multitude of aryl halides can be converted economically to monoarylpiperazines with good chemical yields.

A process has now been found for preparing compounds of the formula (I) 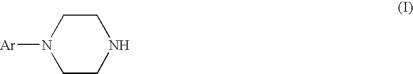
in which
Ar is a mono-, bi- or tricyclic aromatic radical having a total of 5 to 18 ring atoms, where no, one or two ring atom(s) per cycle is/are selected from the group of oxygen, sulphur and nitrogen, and the mono-, bi- or tricyclic aromatic radical is optionally mono- or polysubstituted,
which is characterized in that
   compounds of the formula (II)
      Ar-Hal
in which Ar is as defined above and Hal is chlorine, bromine or iodine
   are reacted with piperazine,
the reaction taking place
   in the presence of palladium complexes which bear, as ligands, compounds of the formula (III) 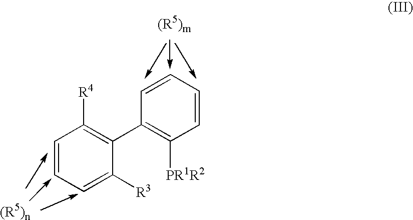
      in which
R¹ and R² are each independently C₁-C₁₂-alkyl or C₅-C₁₁-arylalkyl,
R³ and R⁴ are each independently H, C₁-C₆-alkyl, C₁-C₆-alkoxy or di-C₁-C₆-alkylamino, preferably C₁-C₆-alkyl or C₁-C₆-alkoxy,
the R⁵ radicals are each independently C₁-C₁₂-alkyl, C₁-C₁₂-alkoxy, C₁-C₁₂-fluoroalkyl, C₁-C₁₂-fluoroalkoxy, fluorine, C₄-C₁₀-aryl or C₅-C₁₁-arylalkyl, and the arrows indicate the possible binding points to the particular aryl radical and
n and m are each independently 0, 1, 2, or 3 and
in the presence of alkali metal base.

The scope of the invention encompasses all radical definitions, parameters and illustrations above and listed below, in general or within areas of preference, i.e. also between the particular areas and areas of preference in any combination.

Alkyl and alkoxy are each independently a straight-chain, cyclic, branched or unbranched alkyl and alkoxy radical respectively, and the radicals mentioned may optionally be further substituted by C₁-C₄-alkoxy radicals. The same applies to the alkylene moiety of an arylalkyl radical or to the alkyl moiety of a dialkylamino radical.

C₁-C₆-Alkyl is, for example and with preference, methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, and C₁-C₁₂-alkyl additionally, for example, n-heptyl, n-octyl, n-decyl and n-dodecyl.

C₁-C₁₂-Alkoxy is, for example and with preference, methoxy, ethoxy, n-propoxy, isopropoxy, n-butoxy, sec-butoxy, tert-butoxy, n-pentoxy, cyclohexoxy, cyclopentoxy, n-hexoxy, and C₁-C₁₂-alkoxy additionally, for example, n-heptoxy, n-octoxy, n-decoxy and n-dodecoxy.

Di-C₁-C₆-alkylamino is, for example and with preference, N,N-dimethylamino, N,N-diethylamino or N,N-diisopropylamino.

Fluoroalkyl and fluoroalkoxy are each independently a straight-chain, cyclic, branched or unbranched alkyl radical and alkoxy radical respectively, each of which is singly, multiply or fully substituted by fluorine atoms.

For example, C₁-C₁₂-fluoroalkyl is trifluoromethyl, 2,2,2-trifluoroethyl, pentafluoroethyl, nonafluorobutyl, heptafluoroisopropyl, perfluorooctyl and perfluorododecyl.

For example, C₁-C₁₂-fluoroalkoxy is trifluoromethoxy, 2,2,2-trifluoroethoxy, pentafluoroethoxy, nonafluorobutoxy, heptafluoroisopropoxy, perfluorooctoxy and perfluorododecoxy.

Aryl is in each case a heteroaromatic radical having 5 to 10 skeleton carbon atoms in which one, two or three skeleton carbon atoms per cycle may be substituted by heteroatoms selected from the group of nitrogen, sulphur or oxygen, or preferably a carbocyclic aromatic radical having 6 to 14 skeleton carbon atoms.

The examples of mono-, bi- or tricyclic carbocyclic aromatic radicals having 6 to 10 skeleton carbon atoms are phenyl, biphenyl, naphthyl, phenanthrenyl, anthracenyl or fluoroenyl; mono-, bi- or tricyclic heteroaromatic radicals having 5 to 14 skeleton carbon atoms in which no, one, two or three skeleton carbon atoms per cycle, but at least one skeleton carbon atom in the entire molecule, may be substituted by heteroatoms selected from the group of nitrogen, sulphur or oxygen are, for example, pyridinyl, oxazolyl, benzofuranyl, dibenzofuranyl or quinolinyl.

In addition, the carbocyclic aromatic radical or heteroaromatic radical may be substituted by up to five identical or different substituents per cycle which are selected from the group of fluorine, cyano, C₁-C₁₂-alkyl, C₁-C₁₂-fluoroalkyl, C₁-C₁₂-fluoroalkoxy, C₁-C₁₂-alkoxy or di(C₁-C₈-alkyl)amino.

Arylalkyl is in each case independently a straight-chain, cyclic, branched or unbranched alkyl radical as defined above which may be substituted singly, multiply or fully by aryl radicals as defined above.

The preferred substitution patterns for the formulae (I) and (II) are defined hereinbelow:
Ar is preferably aryl as defined above, more preferably a phenyl or naphthyl radical which is unsubstituted or mono-, di- or trisubstituted by radicals which are selected from the group of nitro, cyano, chlorine, fluorine, C₁-C₁₂-alkyl, C₁-C₁₂-fluoroalkyl, C₁-C₁₂-fluoroalkoxy, C₁-C₁₂-alkoxy, di(C₁-C₈-alkyl)amino, and most preferably a phenyl radical which is unsubstituted or mono-, di- or trisubstituted by radicals which are selected from the group of fluorine, C₁-C₄-alkyl, C₁-C₄-fluoroalkyl, C₁-C₄-fluoroalkoxy or C₁-C₄-alkoxy.
Hal is preferably chlorine or bromine, more preferably chlorine.

A particularly preferred compound of the formula (I) is 4-trifluoromethylphenylpiperazine; a particularly preferred compound of the formula (II) is 4-trifluoromethylchlorobenzene (often also referred to as 4-chlorobenzotrifluoride).

The preferred substitution patterns for formula (m) are defined hereinbelow:
R¹ and R² are preferably each independently isopropyl, tert-butyl, cyclopentyl or cyclohexyl, more preferably each identically the aforementioned radicals and most preferably each identically cyclohexyl.
R³ and R⁴ are preferably each independently H, methyl, ethyl, isopropyl, methoxy or N,N-dimethylamino, more preferably methyl, ethyl, isopropyl or methoxy, most preferably each identically the aforementioned radicals and, in preferred embodiments, each identically isopropyl or methoxy.
n is preferably 0 or 1.
m is preferably 0.
R⁵ is preferably in each case independently methyl, ethyl, isopropyl and methoxy.

Particularly preferred compounds of the formula (III) are [2-(2,4,6-triisopropylphenyl)phenyl]dicyclohexylphosphine and [2-(2,6-dimethoxyphenyl)phenyl]dicyclohexylphosphine.

The compounds of the formula (III) are known from the literature and can be prepared, for example, according to E. R. Strieter, D. G. Blackmond, S. L. Buchwald, Journal of the American Chemical Society, 125, 2003, 13978-13980.

Useful palladium complexes which bear, as ligands, compounds of the formula (III) are, for example, isolated or preformed palladium complexes containing compounds of the formula (III) or those which are obtained by reacting a palladium precursor with compounds of the formula (III) in the reaction medium.

Preference is given to obtaining the palladium complexes used for the process by reacting a palladium precursor with compounds of the formula (III) in the reaction medium.

Suitable palladium precursors are all palladium compounds which can react with compounds of the formula (III) to form palladium-phosphorus coordination.

Preferred palladium precursors are: Pd₂(dibenzylideneacetone)₃ or allylpalladium chloride or bromide or palladium compounds of the formula (IVa)
PdY¹₂ (IVa)
in which
Y¹ is an anion, preferably chloride, bromide, acetate, propionate, nitrate, methanesulphonate, trifluoromethanesulphonate, acetylacetonate, allyl or cyclopentadienyl,
or palladium compounds of the formula (IVb)
   PdY²₂L₂ (Vb)
   in which
Y² is an anion, preferably chloride, bromide, acetate, methanesulphonate or trifluoromethanesulphonate, nonafluorobutanesulphonate, tetrafluoroborate or hexafluorophosphate and
L is in each case a nitrile, preferably acetonitrile, benzonitrile or benzyl nitrile, or an olefin, preferably cyclohexene or cyclooctene, or
L₂ together is a diolefin, preferably norbomadiene or 1,5-cyclooctadiene.

Preferred palladium precursors are palladium(II) acetate and [Pd₂(dba)₃].

The molar ratio of palladium to compounds of the formula (III) may, for example, be 1 to 4, but preferably 1.5 to 2.5 and more preferably 1.5 to 2.2, in particular exactly 2.

The molar ratio of palladium to compounds of the formula (I) may be, for example, 0.000001 to 0.05, but preferably 0.00001 to 0.01 and more preferably 0.0001 to 0.001.

The process according to the invention is carried out in the presence of alkali metal base. Alkali metal bases are, for example and with preference, alkali metal or alkaline earth metal hydroxides or alkoxides, for example lithium, sodium, potassium or calcium hydroxide, sodium or potassium methoxide, ethoxide, n- or i-propoxide, n-, i-, s- or t-butoxide or i-pentoxide; more preferably sodium or potassium hydroxide or tert-butoxide, and most preferably sodium hydroxide.

The molar ratio of alkali metal base to compounds of the formula (I) may, for example, be 1 to 2.2, but preferably 1.2 to 1.6 and more preferably 1.4. Larger amounts are possible but uneconomic.

The molar ratio of piperazine to compounds of the formula (I) may, for example, be 1 to 3, but preferably 1.5 to 2 and more preferably 1.5. Larger amounts are possible but uneconomic.

In a preferred embodiment, the process is also carried out in the presence of organic solvent. Suitable organic solvents are in particular aromatic hydrocarbons, for example benzene, toluene and xylenes; amides, for example N,N-dimethylformamide, N,N-dimethylacetamide, N-methylformanilide, N-methylpyrrolidone and N-methylcaprolactam; ethers, for example diethyl ether, methyl tert-butyl ether, diisopropyl ether, dioxane, tetrahydrofuran or ethylene glycol dimethyl ether or ethylene glycol diethyl ether; alcohols, for example methanol, ethanol, n- or i-propanol, tert-butanol, ethylene glycol monomethyl ether, ethylene glycol monoethyl ether, diethylene glycol monomethyl ether, diethylene glycol monoethyl ether, tertiary amines, for example tri-n-butylamine or triethylamine or mixtures of such organic solvents.

Particularly useful organic solvents have been found to be: mixtures of aromatic hydrocarbons and ethers, in particular a mixture of toluene and tetrahydrofuran, mixtures of aromatic hydrocarbons and alcohols, in particular mixtures of toluene and methanol, and also tertiary amines.

The reaction temperature may be, for example, 30 to 150° C., preferably 60 to 150° C., more preferably 70 to 120° C., the reaction pressure 0.5 to 100 bar, preferably ambient pressure.

In the inventive manner, it is possible to obtain compounds of the formula (I) in high purity and yield in a simple manner.

The advantage of the process according to the invention is in particular that high chemical yields of monoarylpiperazines can be obtained at low amounts of catalyst without having to use a high excess of piperazine.

### EXAMPLES

### Examples 1 to 6

Comparative Experiment

Chlorobenzotrifluoride (18.5 g, 100 mmol) and piperazine (12.9 g) were initially charged in 400 ml of xylene and the mixture was degassed at room temperature by passing nitrogen through for 15 min. Dry sodium tert-butoxide (13.5 g, 140 mmol) was added and the mixture was degassed for a further 10 min. In a separate vessel, tris-tert-butylphosphine (81 mg, 0.4 mmol) with careful exclusion of air and (dibenzylideneacetone)palladium (41 mg, 0.05 mmol) were stirred under nitrogen in 10 ml of degassed tetrahydrofuran. After 30 min, this catalyst solution was introduced dropwise at room temperature into the larger flask with the aid of a transfer needle. On completion of addition, the reaction was heated to internal temperature 120° C. After 8 h, the reaction mixture was allowed to cool to 50° C. and the precipitated solid was filtered off. The solvent was removed under reduced pressure and the crude product isolated by chromatographic purification on silica gel. 12.4 g (54 mmol, 54% of theory) of N-(4-trifluoromethylphenyl)piperazine were obtained.

### Example 1

Chlorobenzotrifluoride (18.5 g, 100 mmol) and piperazine (12.9 g, 150 mmol) were dissolved in a mixture of 120 ml of toluene and 80 ml of tetrahydrofuran, and degassed at room temperature by passing nitrogen through for 15 min. Dry sodium tert-butoxide (13.5 g, 140 mmol) was added and the mixture was degassed for a further 10 min. In a separate vessel, [2-(2,6-dimethoxyphenyl)phenyl]dicyclohexylphosphine (41 mg, 0.1 mmol) and (dibenzylideneacetone)palladium (20 mg, 0.025 mmol) were stirred under nitrogen in 10 ml of degassed tetrahydrofuran. After 30 min, this catalyst solution was introduced dropwise at room temperature into the larger flask with the aid of a transfer needle. On completion of addition, the reaction was heated to internal temperature 90° C. After 8 h, the reaction was allowed to cool to 50° C. and the precipitated solid was filtered off. The filtrate was extracted with dilute hydrochloric acid at pH 3. The aqueous phase was removed and adjusted to pH 10 with the aid of sodium hydroxide solution. The precipitated white solid was filtered off and dried under reduced pressure. 21.4 g (93 mmol, 93% of theory) of N-(4-trifluoromethylphenyl)piperazine were obtained. The product content of the solid was determined to be >99% by quantitative proton NMR.

### Example 2

Chlorobenzotrifluoride (18.5 g, 100 mmol) and piperazine (12.9 g, 150 mmol) were dissolved in a mixture of 120 ml of toluene and 80 ml of tetrahydrofuran, and degassed at room temperature by passing nitrogen through for 15 min. Dry sodium tert-butoxide (13.5 g, 140 mmol) was added and the mixture was degassed for a further 10 min. In a separate vessel, [2-(2,6-dimethoxyphenyl)phenyl]dicyclohexylphosphine (164 mg, 0.4 mmol) and palladium acetate (45 mg, 0.2 mmol) were stirred under nitrogen in 10 ml of degassed tetrahydrofuran. After 30 min, this catalyst solution was introduced dropwise at room temperature into the larger flask with the aid of a transfer needle. On completion of addition, the reaction was heated to internal temperature 90° C. After 8 h, the reaction was allowed to cool to 50° C. and the precipitated solid was filtered off. The filtrate was extracted with dilute hydrochloric acid at pH 3. The aqueous phase was removed and adjusted to pH 10 with the aid of sodium hydroxide solution. The precipitated white solid was filtered off and dried under reduced pressure. 21.8 g (95 mmol, 95% of theory) of N-(4-trifluoromethylphenyl)piperazine were obtained. The product content of the solid was determined to be >99% by quantitative proton NMR.

### Example 3

Chlorobenzotrifluoride (18.5 g, 100 mmol) and piperazine (12.9 g, 150 mmol) were dissolved in a mixture of 120 ml of toluene and 80 ml of methanol, and degassed at room temperature by passing nitrogen through for 15 min. Sodium hydroxide (5.6 g, 140 mmol) was added and the mixture was degassed for a further 10 min. In a separate vessel, [2-(2,4,6-triisopropylphenyl)phenyl]dicyclohexylphosphine (95 mg, 0.2 mmol) and (dibenzylideneacetone)palladium (40 mg, 0.05 mmol) were stirred under nitrogen in 10 ml of degassed tetrahydrofuran. After 30 min, this catalyst solution was introduced dropwise at room temperature into the larger flask with the aid of a transfer needle. On completion of addition, the reaction was heated to internal temperature 78° C. After 8 h, the reaction was allowed to cool to 50° C. and the precipitated solid was filtered off. The filtrate was extracted with dilute hydrochloric acid at pH 3. The aqueous phase was removed and adjusted to pH 10 with the aid of sodium hydroxide solution. The precipitated white solid was filtered off and dried under reduced pressure. 21.8 g (95 mmol, 95% of theory) of N-(4-trifluoromethylphenyl)piperazine were obtained. The product content of the solid was determined to be >99% by quantitative proton NMR.

### Example 4

Chlorobenzotrifluoride (18.5 g, 100 mmol) and piperazine (12.9 g, 150 mmol) were dissolved in 200 ml of tri-n-butylamine, and degassed at room temperature by passing nitrogen through for 15 min. Dry sodium tert-butoxide (13.5 g, 140 mmol) was added and the mixture was degassed for a further 10 min. In a separate vessel, [2-(2,6-dimethoxyphenyl)phenyl]dicyclohexylphosphine (41 mg, 0.1 mmol) and (dibenzylideneacetone)palladium (20 mg, 0.025 mmol) were stirred under nitrogen in 5 ml of degassed tetrahydrofuran. After 30 min, this catalyst solution was introduced dropwise at room temperature into the larger flask with the aid of a transfer needle. On completion of addition, the reaction was heated to internal temperature 110° C. After 8 h, the reaction was allowed to cool to 50° C. and the precipitated solid was filtered off. The filtrate was concentrated under reduced pressure and extracted with dilute hydrochloric acid at pH 3. The aqueous phase was removed and adjusted to pH 10 with the aid of sodium hydroxide solution. The precipitated white solid was filtered off and dried under reduced pressure. 22.1 g (96 mmol, 96% of theory) of N-(4-trifluoromethylphenyl)piperazine were obtained. The product content of the solid was determined to be >99% by quantitative proton NMR.

### Example 5

Bromobenzotrifluoride (22.5 g, 100 mmol) and piperazine (12.9 g, 150 mmol) were dissolved in 200 ml of toluene, and degassed at room temperature by passing nitrogen through for 15 min. Dry sodium tert-butoxide (13.5 g, 140 mmol) was added and the mixture was degassed for a further 10 min. In a separate vessel, [2-(2,4,6-triisopropylphenyl)phenyl]dicyclohexylphosphine (95 mg, 0.2 mmol) and palladium acetate (23 mg, 0.1 mmol) were stirred under nitrogen in 10 ml of degassed tetrahydrofuran. After 30 min, this catalyst solution was introduced dropwise at room temperature into the larger flask with the aid of a transfer needle. On completion of addition, the reaction was heated to internal temperature 110° C. After 8 h, the reaction was allowed to cool to 50° C. and the precipitated solid was filtered off. The filtrate was extracted with dilute hydrochloric acid at pH 3. The aqueous phase was removed and adjusted to pH 10 with the aid of sodium hydroxide solution. The precipitated white solid was filtered off and dried under reduced pressure. 22.1 g (96 mmol, 96% of theory) of N-(4-trifluoromethylphenyl)piperazine were obtained. The product content of the solid was determined to be >99% by quantitative proton NMR.

### Example 6

4-Cyanochlorobenzene (13.8 g, 100 mmol) and piperazine (12.9 g, 150 mmol) were dissolved in a mixture of 120 ml of toluene and 80 ml of tetrahydrofuran, and degassed at room temperature by passing nitrogen through for 15 min. Dry sodium tert-butoxide (13.5 g, 140 mmol) was added and the mixture was degassed for a further 10 min. In a separate vessel, [2-(2,4,6-triisopropylphenyl)phenyl]dicyclohexylphosphine (95 mg, 0.2 mmol) and (dibenzylideneacetone)palladium (40 mg, 0.05 mmol) were stirred under nitrogen in 10 ml of degassed tetrahydrofuran. After 30 min, this catalyst solution was introduced dropwise at room temperature into the larger flask with the aid of a transfer needle. On completion of addition, the reaction was heated to internal temperature 90° C. After 8 h, the reaction was allowed to cool to 50° C. and the precipitated solid was filtered off. The filtrate was extracted with dilute hydrochloric acid at pH 3. The aqueous phase was removed and adjusted to pH 10 with the aid of sodium hydroxide solution. The precipitated white solid was filtered off and dried under reduced pressure. 17.8 g (95 mmol, 95% of theory) of N-(4-cyanophenyl)piperazine were obtained. The product content of the solid was determined to be >99% by quantitative proton NMR.

1. Process for preparing compounds of the formula (I) 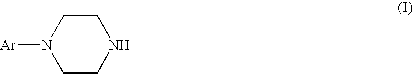
in which
Ar is a mono-, bi- or tricyclic aromatic radical having a total of 5 to 18 ring atoms, where no or one ring atom per cycle is selected from the group of oxygen, sulphur and nitrogen, and the mono-, bi- or tricyclic aromatic radical is optionally mono- or polysubstituted,
characterized in that
compounds of the formula (II)
Ar-Hal (II)
in which Ar is as defined above and Hal is chlorine, bromine or iodine
are reacted with piperazine,
the reaction taking place
in the presence of palladium complexes which bear, as ligands, compounds of the formula (III) 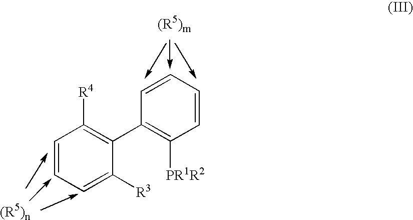
in which
R¹ and R² are each independently C₁-C₁₂-alkyl or C₅-C₁₁-arylalkyl,
R³ and R⁴ are each independently H, C₁-C₆-alkyl, C₁-C₆-alkoxy or di-C₁-C₆-alkylamino,
the R⁵ radicals are each independently C₁-C₁₂-alkyl, C₁-C₁₂-alkoxy, C₁-C₁₂-fluoroalkyl, C₁-C₁₂-fluoroalkoxy, fluorine, C₄-C₁₀-aryl or C₅-C₁₁-arylalkyl, and the arrows indicate the possible binding points to the particular aryl radical and
n and m are each independently 0, 1, 2, or 3 and
in the presence of alkali metal base.

2. Process according to claim 1, characterized in that the palladium complexes which bear, as ligands, compounds of the formula (III) are those which are obtained by reacting a palladium precursor with compounds of the formula (III) in the reaction medium.

3. Process according to claim 1 or 2, characterized in that the molar ratio of palladium to compounds of the formula (III) is 1 to 4.

4. Process according to claim 1, characterized in that the molar ratio of palladium to compounds of the formula (I) is 0.000001 to 0.05.

5. Process according to claim 1, characterized in that the alkali metal bases used are alkali metal or alkaline earth metal hydroxides or alkoxides.

6. Process according to claim 1, characterized in that the molar ratio of piperazine to compounds of the formula (I) is 1.2 to 1.8.

7. Process according to claim 1, characterized in that the process is also carried out in the presence of organic solvents.

8. Process according to claim 7, characterized in that the organic solvents used are aromatic hydrocarbons, amides, ethers, alcohols, tertiary amines or mixtures of such organic solvents.
